(19) United States
(12) Patent Application Publication
(10) Pub. No.: US 2023/0094265 A1
(43) Pub. Date: Mar. 30, 2023

(54) COMPOSITIONS FOR HEMORRHOID TREATMENT

(71) Applicant: INNOCAN PHARMA LTD., Herzliya (IL)

(72) Inventor: Nir AVRAM, Metar (IL)

(73) Assignee: INNOCAN PHARMA LTD., Herzliya (IL)

(21) Appl. No.: 17/795,553

(22) PCT Filed: Jan. 20, 2021
(86) PCT No.: PCT/IL2021/050059

§ 371 Date: Jul. 27, 2022

Related U.S. Application Data
(60) Provisional application No. 62/967,614, filed on Jan. 30, 2020.

(51) Int. Cl. D04H 1/4266; A61K 9/107; A61K 36/882; A61K 36/73; A61K 31/05; A61K 36/14; A61K 36/534; A61K 31/5375; A61K 9/70

(52) U.S. Cl. CPC D04H 1/4266; A61K 9/107; A61K 36/882; A61K 36/73; A61K 31/05; A61K 36/14; A61K 36/534; A61K 31/5375; A61K 9/7007

(57) ABSTRACT

Described herein are compositions in the form of wet wipes comprising active agents to treat hemorrhoids. The compositions comprise a fabric, a cannabinoid and at least one additional agent, the agent preferably selected from the group consisting of a vasoconstrictor and an astringent. Additionally described herein are methods for treating hemorrhoids comprising applying to an affected area of a person in need thereof a wet wipe comprising a fabric, a cannabinoid and at least one additional agent the agent preferably selected from the group consisting of a vasoconstrictor and an astringent.

### CROSS REFERENCE TO RELATED APPLICATIONS

Benefit is claimed to U.S. Provisional Patent Application 62/967,614 filed Jan. 30, 2020; the contents of which is incorporated by reference herein in its entirety.

### FIELD

Provided herein are compositions for the treatment of hemorrhoids.

### BACKGROUND

Hemorrhoids are swollen veins in the anus and lower rectum, which lead to itching, irritation, pain, swelling and/or bleeding. Hemorrhoids can be external, in the anal area, or internal, inside the rectum.

Hemorrhoids are caused by the bulging, stretching or swelling of veins in the anorectal area. The underlying cause of hemorrhoids may be straining while passing bowel movements, constipation, pregnancy, obesity, or lifting heavy weights.

### SUMMARY

Described herein are compositions in the form of wet wipes comprising active agents to treat hemorrhoids. The compositions comprise a fabric, a cannabinoid and at least one additional agent, the agent preferably selected from the group consisting of a vasoconstrictor and an astringent.

Additionally described herein are methods for treating hemorrhoids comprising applying to an affected area of a person in need thereof a wet wipe comprising a fabric, a cannabinoid and at least one additional agent the agent preferably selected from the group consisting of a vasoconstrictor and an astringent.

The foregoing and other objects, features, and advantages will become more apparent from the following detailed description.

### DETAILED DESCRIPTION

Unless otherwise noted, technical terms are used according to conventional usage. Definitions of common terms in pharmaceutical sciences can be found in Troy et al. Remington: The Science and Practice of Pharmacy. Published by Lippincott Williams & Wilkins, 2006. In case of conflict, the present specification, including explanations of terms, will control. In addition, all the materials, methods, and examples are illustrative and not intended to be limiting.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia and is used herein to indicate a non-limiting example. Thus, the abbreviation “e.g.” is synonymous with the term “for example.”

As mentioned above, embodiments relate to wet wipes. Wet wipes comprise a fabric and a liquid composition embedded in the wet wipe. The liquid compositions comprise a cannabinoid, in combination with at least one additional active ingredient, in the form of a wet wipe. According to an embodiment, the active ingredient is an astringent or a vasoconstrictor. Optionally, the active ingredient is an anesthetic. The astringent may be witch hazel. The vasoconstrictor may be phenylephrine. According to an embodiment, the anesthetic is pramoxine. Without being bound by theory, it is suggested that cannabinoids will act synergistically with at least one additional active ingredient as described herein to provide an effect to enhance treatment of hemorrhoids.

Witch hazel is an astringent formed as an extract from the plant Hamamelis virginiana. Astringents are agents which cause contraction of human tissue when applied. Additional or alternative astringents which may be used include, but are not limited to: calamine and tannic acid.

Phenylephrine is a vasoconstrictor which acts as an α₁-adrenergic receptor activator. Vasoconstrictors are agents which reduce blood flow by narrowing blood vessels. When applied locally, for example, topically or rectally, vasoconstrictors can treat affected areas by limiting blood flow to the applied area.

Additional or alternative vasoconstrictors which may be used include: ephedrine sulfate, epinephrine, or salts thereof.

Pramoxine is an anesthetic and antipruritic which may be used for topical, rectal and/or vaginal administration. Local administration of an anesthetic reduces sensation of pain and/or itching at the site of local administration.

Additional or alternative anesthetics which may be used include, but are not limited to camphor, juniper tar, menthol, diperodon, phenacaine, benzocaine, benzyl alcohol, dibucaine, dyclonine, lidocaine, and tetracaine, and salts thereof.

According to an embodiment, an astringent, for example, witch hazel, may be present in an amount between 10% and 70% of the liquid composition. Optionally, the astringent may be present in an amount between 40% and 60% of the liquid composition. Optionally, the astringent is present in an amount of 50% of the liquid composition.

According to an embodiment, a vasoconstrictor, for example phenylephrine, may be present in an amount between about 0.1% and about 1% of the liquid composition. Optionally, the vasoconstrictor may be present in an amount between 0.2% and 0.5% of the liquid composition. Optionally, the vasoconstrictor may be present in an amount of 0.25% of the liquid composition.

According to an embodiment, an anesthetic, for example pramoxine, may be present in an amount between about 0.1% and about 2% of the liquid composition. Optionally, the anesthetic may be present in an amount between 0.5% and 1.5% of the liquid composition. Optionally, the anesthetic may be present in an amount of 1% of the liquid composition.

A cannabinoid is a chemical compound that acts on cannabinoid receptors in cells in mammals, including in humans. Cannabinoids can be manufactured synthetically or obtained from various parts of the genus Cannabis, in particular, from the species Cannabis Sativa. Two preferred cannabinoids according to various embodiments, are (−)-trans-Δ⁹-tetrahydrocannabinol, and/or isomers thereof (THC) and cannabidiol (CBD). Alternatively, a cannabinoid may be in the form of hemp oil. Alternatively, a cannabinoid may be in the form of cannabis oil or isolate. Compositions described herein may comprise one cannabinoid or multiple cannabinoids, such as a combination of CBD and THC. Cannabinoids may be synthetically produced or isolated from plants.

According to an embodiment, a cannabinoid may be present in an amount between about 0.01% and about 10% of the liquid composition. Optionally, a cannabinoid may be present in an amount between about 0.1% and 1% of the liquid composition. Optionally, the cannabinoid may be present in an amount between 0.25% and 0.5% of the liquid composition, preferably 0.35% of the composition.

Additional active ingredients may be selected from the group consisting of analgesics, or antipruritic agents. Examples of analgesics and antipruritic agents include camphor, juniper tar and menthol.

In addition to active ingredients, compositions described herein may further comprise at least one inert ingredient. The inert ingredient may be selected from the group consisting of: water, a solvent, an emulsifier, an emollient, a moisturizer, a pH adjustment agent, a polymer, a humectant, an occlusive agent, a preservative, a thickener, an anti-irritation agent, a conditioning agent, a buffer, a vitamin, an extract, a natural oil, a wax, a penetration enhancer, a peptide, a sugar derivative, a fatty acid, a fatty alcohol, a silicone, a polyethyl-glycol, a fragrance, a pigment, an ester, a triglyceride, a butter, hyaluronic acid, and an absorbing powder.

The preservative may be selected from the group consisting of: a parahydroxybenzoic acid, methylparaben, propylparaben, Benzyl Alcohol, Phenoxyethanol, Ethylhexylglycerin, Octanediol, Hexanediol, Pentandiol, Sorbitan Caprylate, capralyl glycol, caprylhydroxamic acid, Phenoxyethanol Triethylene glycol, sodium benzoate, and Bronopol. Preferably, the preservative is Phenoxyethanol Triethylene glycol.

Optionally the liquid compositions further comprise a moisturizer. The moisturizer may be, for example, aloe barbadensis leaf juice or panthenol.

Optionally, the liquid compositions further comprise a humectant. The humectant may be glycerin. The humectant may be present in the liquid composition in an amount of between 1% and 5%, preferably 3%.

Optionally, the liquid composition may be an emulsion, such as an oil in water emulsion. Without being bound by theory, it is suggested that improved moisturizing of the affected area can be obtained in patients suffering from hemorrhoids using an oil in water emulsion liquid composition in a wet wipe when compared to an aqueous-based wet wipe.

In order to form emulsions, liquid compositions may be prepared with at least one emulsifier. Optionally, the emulsifier is selected from the group consisting of: Acrylates/C10-30 alkyl acrylate crosspolymer (Pemulen™ Tr-2); Sodium Acrylates/C10-30 Alkyl Acrylates Crosspolymers (Cosmedia® SP); Trilaureth-4 Phosphate (Hostaphat® KL 340) and Cetearyl Isononanoate (and) Ceteareth-20 (and) Cetearyl Alcohol (and) Glyceryl Stearate (and) Glycerin (and) Ceteareth-12 (and) Cetyl Palmitate (Emulgade® CM). Optionally, the emulsifier is selected from the group consisting of: C14-22 Alcohols (and) C12-20 Alkyl Glucoside (MONTANOV L); Arachidyl Alcohol (and) Behenyl Alcohol (and) Arachidyl Glucoside (MONTANOV 202); Polyacrylate Crosspolymer-6 (SEPIMAX ZEN); Microcrystalline cellulose and cellulose gum (AVICEL PC 611); Sucrose Palmitate (Sisterna PS750-C); Aqua, Sucrose Laurate, Alcohol (Sisterna L70-C); CETEARYL ALCOHOL AND CETETH-20 PHOSPHATE AND DICETYL PHOSPHAT (CRODAFOS CS20 ACID); Potassium Laureth-1 Phosphate (Dermalcare® MAP L-213/K); Hydroxyethyl Acrylate/Sodium Acryloyldimethyl Taurate Copolymer (and) Isohexadecane (and) Polysorbate 60 (SIMULGEL™ INS 100); Glyceryl Stearate Citrate (and) Polyglyceryl-3 Stearate (and) Hydrogenated Lecithin (Heliofeel™); Ceteareth-25 (and) Disodium Ethylene Dicocamide PEG-15 Disulfate (CERALUTION ES RSPO-MB); Sorbitan Stearate (SPAN 60); Glyceryl Stearate (and) PEG-100 Stearate (ARLACEL 165 FL); Polyacrylate Crosspolymer-11 (Aristoflex® Velvet); Ammonium Acryloyldimethyltaurate/Beheneth-25 Methacrylate Crosspolymer (Aristoflex® BLV); Ammonium Acryloyldimethyltaurate/VP Copolymer; Glyceryl Stearate; (Aristoflex® AVC); (Aristoflex® TAC); Ammonium Acryloyldimethyltaurate/Carboxyethyl Acrylate Crosspolymer (Aristoflex® TAC); Acrylates/C10-30 Alkyl Acrylate Crosspolymer (Carbopol® Ultrez 20 Polymer); Acrylates Copolymer (Carbopol® Aqua SF-1 Polymer); Lauryl Glucoside (and) Polyglyceryl-2 Dipolyhydroxystearate (and) Glycerin (Eumulgin® VL 75); and CETEARETH-22 (and) PALMETH-2 (EMULPHARMA XL).

Optionally, the ratio in the wet wipes between fabric and liquid composition is between 1:2 and 1:4, preferable 1:3.

The wet wipes according embodiments may be individually packaged in a single-wipe packet. Alternatively, wipes may be packaged in a container, such as a bottle or resealable bag. The wipes may be packaged in a flow pack so that when a first wipe is removed, a subsequent wipe is partially advanced to the opening of the flow pack.

As described herein, certain embodiments relate to methods for treatment of hemorrhoids comprising applying to an affected are of a person in need thereof a wet wipe comprising a comprise a fabric, a cannabinoid and at least one additional agent. Optionally, the hemorrhoids may be external hemorrhoids.

According to an embodiment, the composition may be applied between once and 6 times daily. Optionally the composition may be applied after the patient in need thereof has a bowel movement.

Wet wipes described herein are advantageous in that they combine ingredients that are water soluble, such as astringent agents, vasoconstrictors and/or anesthetic agents, in combination with active cannabinoids which are not soluble in water.

The following examples are provided to illustrate certain particular features and/or embodiments. These examples should not be construed to limit the disclosure to the particular features or embodiments described.

### EXAMPLES

### Example 1A. Preparation of Wet Wipes with CBD and Witch Hazel

A liquid for preparing wet wipes is prepared according to the ingredients in Table 1.

TABLE 1

| | | Amount |
|---|---|---|
| Ingredient | Function | (weight percent) |
| Water | Aqueous solvent | 39.88 |
| Glycerin 99.7% | Humectant | 3.0 |
| Acrylates/C10-30 alkyl | Emulsifier | 0.175 |
| acrylate crosspolymer | | |
| (Pemulen ™ Tr-2) | | |
| Sodium Hydroxide (20%) | pH control | 0.3 |
| Aloe Barbadensis Leaf | Skin moisturizing/ | 2.0 |
| Juice | soothing | |
| Witch Hazel | Soothing, pain relief, | 50.0 |
| | astringent | |
| Sativa (Cannabis) Seed oil | Solvent for CBD | 3.0 |
| (Hemp oil) | | |
| Cannabidiol | Pain relief | 0.35 |
| Bisabolol | Skin penetration agent | 0.1 |
| Polysorbate-20 | Emuslifier | 0.2 |
| Phenoxyethanol and | Preservative | 1.0 |
| triethylene glycol | | |
| (Iscaguard ® PEG) | | |
| Total | | 100 |

Pemulen TR-2 is provided by Lubrizol Corp. Wickliffe, Ohio, USA. Iscaguard is provided by ISCA UK Ltd., United Kingdom. Cannabidiol is Hemp-derived CBD Isolate, having a purity of >97%.

Glycerin is added to Acrylates/C10-30 alkl acrylate crosspolymer, and homogenized until a clear gel is obtained without lumps. NaOH solution is added while mixing until a pH of 6.0-6.5 is obtained. Witch Hazel is then added.

In a separate vessel CBD is added to hemp oil, which is heated to 40° C., until CBD is completely dissolved. The hemp oil is then added to the witch hazel vessel. Bisabolol, Polysorbate-20 and Iscaguard and homogenized for two minutes.

A whitish/translucent liquid is formed.

### Example 1B: Preparation of Wet Wipes with CBD, Witch Hazel, and Phenylephrine HCl

A liquid for preparing wet wipes is prepared according to the ingredients in Table 2.

TABLE 2

| | | Amount |
|---|---|---|
| Ingredient | Function | (weight percent) |
| Water | Aqueous solvent | 39.63 |
| Glycerin 99.7% | Humectant | 3.0 |
| Acrylates/C10-30 alkyl | Emulsifier | 0.18 |
| acrylate crosspolymer | | |
| (Pemulen ™ Tr-2) | | |
| Sodium Hydroxide (20%) | pH control | 0.3 |
| Aloe Barbadensis Leaf | Skin moisturizer/ | 2.0 |
| Juice | soothing | |
| Witch Hazel | Soothing, pain relief, | 50.0 |
| | astringent | |
| Phenylephrine HCl | Pain relief | 0.25 |
| Sativa (Cannabis) Seed oil | Solvent for CBD | 3.0 |
| (Hemp oil) | | |
| Cannabidiol | Pain relief | 0.35 |
| Bisabolol | Skin penetration agent | 0.1 |
| Polysorbate-20 | Emuslifier | 0.2 |
| Phenoxyethanol and | Preservative | 1.0 |
| triethyleneglycol | | |
| (Iscaguard ® PEG) | | |
| Total | | 100 |

Cannabidiol is Hemp-derived CBD Isolate, having a purity of >97%.

Glycerin is added to Acrylates/C10-30 alkyl acrylate crosspolymer, and homogenized until a clear gel is obtained without lumps. NaOH solution is added while mixing until a pH of 6.0-6.5 is obtained. Witch Hazel is then added, then phenylephrine HCl.

In a separate vessel CBD is added to hemp oil, which is heated to 40° C., until CBD is completely dissolved. The hemp oil is then added to the witch hazel vessel. Bisabolol, Polysorbate-20 and Iscaguard and homogenized for two minutes.

A whitish/translucent liquid is formed.

### Example 1C: Preparation of Wet Wipes with CBD and Witch Hazel

A liquid for preparing wet wipes is prepared according to the ingredients in Table 3.

TABLE 3

| | | Amount |
|---|---|---|
| Ingredient | Function | (weight percent) |
| Water | Aqueous solvent | 38.35 |
| Glycerin 99.7% | Humectant | 3.0 |
| Aloe Barbadensis Leaf | Skin moisturizer/ | 2.0 |
| Juice | soothing | |
| Witch Hazel | Soothing, pain relief, | 50.0 |
| | astringent | |
| Sativa (Cannabis) Seed oil | Solvent for CBD | 3.0 |
| (Hemp oil) | | |
| Cannabidiol | Pain relief | 0.35 |
| Trilaureth-4 Phosphate | Emulsifier | 2.0 |
| Bisabolol | Skin penetration agent | 0.1 |
| Polysorbate-20 | Emuslifier | 0.2 |
| Phenoxyethanol and | Preservative | 1.0 |
| triethyleneglycol | | |
| (Iscaguard ® PEG) | | |
| Total | | 100 |

Cannabidiol is Hemp-derived CBD Isolate, having a purity of >97%.

Trilaureth-4 Phosphate is obtained as Hostaphat® KL 340 by Clairant International Ltd., Switzerland.

In water, glycerin is added to aloe. Witch Hazel is then added and mixed.

In a separate vessel CBD is added to hemp oil, which is heated to 40° C., until CBD is completely dissolved. The hemp oil is then added to the aqueous mixture. Trilaureth-4 Phosphate, Bisabolol, Polysorbate-20 and Iscaguard are added and homogenized for 10 minutes.

A whitish/translucent liquid is formed.

### Example 1D: Preparation of Wet Wipes with CBD and Witch Hazel

Preparation of wet wipes with CBD and witch hazel.
A liquid for preparing wet wipes is prepared according to the ingredients in Table 4.

TABLE 4

| | | Amount |
|---|---|---|
| Ingredient | Function | (weight percent) |
| Water | Aqueous solvent | 40.18 |
| Glycerin 99.7% | Humectant | 3.0 |
| Cosmedia ® SP | Emulsifier | 0.18 |
| Aloe Barbadensis Leaf | Skin moisturizer/ | 2.0 |
| Juice | soothing | |
| Witch Hazel | Soothing, pain relief, | 50.0 |
| | astringent | |
| Sativa (Cannabis) Seed oil | Solvent for CBD | 3.0 |
| (Hemp oil) | | |
| Cannabidiol | Pain relief | 0.35 |
| Bisabolol | Skin penetration agent | 0.1 |
| Polysorbate-20 | Emuslifier | 0.2 |
| Phenoxyethanol and | Preservative | 1.0 |
| triethyleneglycol | | |
| (Iscaguard ® PEG) | | |
| Total | | 100 |

Cosmedia® SP is a trade name of BASF, Ludwigshafen, Germany, relating to the product Sodium polyacrylate, Sodium Acrylates/C10-30 Alkyl Acrylates Crosspolymers.

Glycerin is added to Cosmedia® SP and homogenized until a clear gel is obtained without lumps. Witch hazel is then added.

In a separate vessel CBD is added to hemp oil, which is heated to 40° C., until CBD is completely dissolved. The hemp oil is then added to the witch hazel vessel. Bisabolol, Polysorbate-20 and Iscaguard are then added and homogenized for two minutes.

A whitish/translucent liquid is formed.

### Example 2A: Preparation of Wet Wipes with CBD, Phenylephrine HCl, and Pramoxine HCl

A liquid for preparing wet wipes is prepared according to the ingredients in Table 5.

TABLE 5

| Ingredient | Function | Amount (weight percent) |
|---|---|---|
| Water | Aqueous solvent | 84.6 |
| Disodium EDTA | Chelating agent | 0.1 |
| Glycerine | Humectant | 3.00 |
| Hydroxyethylcellulose | | 0.3 |
| (Natrosol ™ 250HHR) | | |
| Sodium Citrate | pH buffer | 0.2 |
| Panthenol | Moisturizer | 0.1 |
| Aloe Barbadensis Leaf | Skin moisturizer/ | 1.0 |
| Juice | soothing | |
| Phenylephrine HCl | Vasoconstrictor | 0.25 |
| Pramoxine HCl | Local anesthetic | 1.0 |
| Emulgade ® CM | Emulsifier | 5.0 |
| Sativa (Cannabis) Seed oil | Solvent for CBD | 3.0 |
| (Hemp Oil) | | |
| Cannabidiol | Pain relief | 0.35 |
| Tocopheryl acetate | Antioxidant | 0.1 |
| Phenoxyethanol and | Preservative | 1.0 |
| triethyleneglycol | | |
| (Iscaguard ® PEG) | | |
| Total | | 100 |

Emulgade® CM is a trade name of BASF, Ludwigshafen, Germany, relating to the product: Cetearyl Isononanoate (and) Ceteareth-20 (and) Cetearyl Alcohol (and) Glyceryl Stearate (and) Glycerin (and) Ceteareth-12 (and) Cetyl Palmitate. Natrosol™ is available from Ashland Global Specialty Chemicals Inc., Covington, Ky., USA.

Water and the subsequent 8 ingredients are added, one by one, and mixed to form a clear solution. In a separate container, Emulgade® CM, hemp oil, cannabidiol, tocopheryl acetate and Iscaguard® are added and mixed to form a clear oil. The cannabidiol containing solution is then mixed with the aqueous solution with moderate mixing to form a whitish lotion with low viscosity.

### Example 2B: Preparation of Wet Wipes with CBD, Phenylephrine HCl, and Pramoxine HCl

A liquid for preparing wet wipes is prepared according to the ingredients in Table 6.

TABLE 6

| Ingredient | Function | Amount (weight percent) |
|---|---|---|
| Water | Aqueous solvent | 82.9 |
| Disodium EDTA | Chelating Agent | 0.1 |
| Glycerine | Humectant | 3.00 |
| Sodium Citrate | pH buffer | 0.2 |
| Panthenol | Moisturizer | 0.1 |
| Aloe Barbadensis Leaf | Skin moisturizer/ | 1.0 |
| Juice | soothing | |
| Phenylephrine HCl | Vasoconstrictor | 0.25 |
| Pramoxine HCl | Anesthetic | 1.0 |
| Emulgade ® CM | Emulsifier | 5.0 |
| Trilaureth-4 Phosphate | Emulsifier | 2.0 |
| Sativa (Cannabis) Seed oil | Solvent for CBD | 3.0 |
| (Hemp Oil) | | |
| Cannabidiol | Pain relief | 0.35 |
| Tocopheryl acetate | Antioxidant | 0.1 |
| Phenoxyethanol and | Preservative | 1.0 |
| triethylene glycol | | |
| (Iscaguard ® PEG) | | |
| Total | | 100 |

Water and the subsequent 6 ingredients are added, one by one, and mixed to form a clear solution.

Emulgade® CM and trilaureth-4 phosphate are mixed, then added to the aqueous solution.

In a separate container, hemp oil, cannabidiol, are added and mixed to form a clear oil. The cannabidiol containing solution is then mixed with the aqueous solution with moderate mixing. Then the tocopheryl acetate and Iscaguard® preservative are added to form a whitish lotion with low viscosity.

### Example 3: Incorporation of Liquid into Wet Wipes

Liquids for wet wipes is prepared according to Examples 1A-D and 2A-B.
Fabric used for the manufacture of wet wipes is optionally non-woven fabric. Optionally, the non-woven fabric is spun-laced fabric. Optionally, the wet wipes are made from one of or a combination of polyester, polypropylene, viscose, Tencel, wood pulp, or cotton. Optionally, the wet wipe fabric comprises polyester and viscose.

The liquid is introduced onto a fabric and the combined product has a ratio of 1:2-1:4, fabric to liquid.

Fabric is shortly thereafter introduced into a sterile receptacle comprising the liquid. The wipes are then cut and folded and packaged into a sealed, waterproof packaging to avoid evaporation or contamination.

According to embodiments, described herein is a wet wipe composition comprising a fabric, and a liquid composition, the liquid composition comprising a cannabinoid and at least one additional active agent selected from the group consisting of a vasoconstrictor and an astringent. Optionally, the composition further comprising an anesthetic. Optionally, the astringent is selected from the group consisting of: witch hazel, calamine and tannic acid. Optionally, the astringent is witch hazel, and is present in an amount of between 10% and 70% of the liquid composition. Optionally, the witch hazel is present in an amount of between 40% and 60% of the liquid composition, optionally, in an amount of 50% of the liquid composition. Optionally, the vasoconstrictor is selected from the group consisting of phenylephrine, ephedrine sulfate, epinephrine, or salts thereof. Optionally, the vacoconstrictor is phenylephrine, and is present in an amount between 0.1% and 1% of the liquid composition. Optionally, phenylephrine is present in an amount between 0.2% and 0.5% of the liquid composition. Optionally, phenylephrine is present in an amount of 0.25% of the liquid composition. Optionally, the anesthetic is selected from the group consisting of: pramoxine, camphor, juniper tar, menthol, diperodon, phenacaine, benzocaine, benzyl alcohol, dibucaine, dyclonine, lidocaine, and tetracaine, and salts thereof. Optionally, the anesthetic is pramoxine, and is present in an amount between 0.1% and 2% of the liquid composition. Optionally, the pramoxine is present in an amount between 0.5% and 1.51% of the liquid composition. Optionally, the pramoxine is present in an amount of 1% of the liquid composition. Optionally, the cannabinoid is cannabidiol (CBD). Optionally, CBD is present in an amount between 0.01% and 10% of the liquid composition, in an amount between 0.1% and 1% of the liquid composition, in an amount between 0.25% and 0.5% of the liquid composition, or in an amount of 0.35% of the liquid composition. Optionally, the composition further comprises an analgesic or antipruritic agent. Optionally, the analgesic or antipruritic agent is selected from the group consisting of: camphor, juniper tar and menthol. Optionally, the composition further comprises one or more of the following ingredients: water, a solvent, an emulsifier, an emollient, a moisturizer, a pH adjustment agent, a polymer, a humectant, an occlusive agent, a preservative, a thickener, an anti-irritation agent, a conditioning agent, a buffer, a vitamin, an extract, a natural oil, a wax, a penetration enhancer, a peptide, a sugar derivative, a fatty acid, a fatty alcohol, a silicone, a polyethyl-glycol, a fragrance, a pigment, an ester, a triglyceride, a butter, hyaluronic acid, and an absorbing powder. Optionally, the moisturizer is selected from the group consisting of aloe barbadensis leaf juice and panthenol. Optionally, the humectant is glycerin. Optionally, the liquid is an oil in water emulsion. Optionally, the emulsifier is selected from the group consisting of: Acrylates/C10-30 alkyl acrylate crosspolymer (Pemulen™ Tr-2); Sodium Acrylates/C10-30 Alkyl Acrylates Crosspolymers (Cosmedia® SP); Trilaureth-4 Phosphate (Hostaphat® KL 340) and Cetearyl Isononanoate (and) Ceteareth-20 (and) Cetearyl Alcohol (and) Glyceryl Stearate (and) Glycerin (and) Ceteareth-12 (and) Cetyl Palmitate (Emulgade® CM); C14-22 Alcohols (and) C12-20 Alkyl Glucoside (MONTANOV L); Arachidyl Alcohol (and) Behenyl Alcohol (and) Arachidyl Glucoside (MONTANOV 202); Polyacrylate Crosspolymer-6 (SEPIMAX ZEN); Microcrystalline cellulose and cellulose gum (AVICEL PC 611); Sucrose Palmitate (Sisterna PS750-C); Aqua, Sucrose Laurate, Alcohol (Sisterna L70-C); CETEARYL ALCOHOL AND CETETH-20 PHOSPHATE AND DICETYL PHOSPHAT (CRODAFOS CS20 ACID); Potassium Laureth-1 Phosphate (Dermalcare® MAP L-213/K); Hydroxyethyl Acrylate/Sodium Acryloyldimethyl Taurate Copolymer (and) Isohexadecane (and) Polysorbate 60 (SIMULGEL™ INS 100); Glyceryl Stearate Citrate (and) Polyglyceryl-3 Stearate (and) Hydrogenated Lecithin (Heliofeel™); Ceteareth-25 (and) Disodium Ethylene Dicocamide PEG-15 Disulfate (CERALUTION ES RSPO-MB); Sorbitan Stearate (SPAN 60); Glyceryl Stearate (and) PEG-100 Stearate (ARLACEL 165 FL); Polyacrylate Crosspolymer-11 (Aristoflex® Velvet); Ammonium Acryloyldimethyltaurate/Beheneth-25 Methacrylate Crosspolymer (Aristoflex® BLV); Ammonium Acryloyldimethyltaurate/VP Copolymer; Glyceryl Stearate; (Aristoflex® AVC); (Aristoflex® TAC); Ammonium Acryloyldimethyltaurate/Carboxyethyl Acrylate Crosspolymer (Aristoflex® TAC); Acrylates/C10-30 Alkyl Acrylate Crosspolymer (Carbopol® Ultrez 20 Polymer); Acrylates Copolymer (Carbopol® Aqua SF-1 Polymer); Lauryl Glucoside (and) Polyglyceryl-2 Dipolyhydroxystearate (and) Glycerin (Eumulgin® VL 75); and CETEARETH-22 (and) PALMETH-2 (EMULPHARMA XL). Optionally, the ratio between fabric and liquid composition is between 1:2 and 1:4. Optionally, the ratio between fabric and liquid composition is 1:3.

Further embodiments relate to a method for treatment of hemorrhoids comprising applying to an affect area of a subject in need thereof, a wet wipe as described above. Optionally, the subject suffers from an external hemorrhoid.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A wet wipe composition comprising a fabric, and a liquid composition, the liquid composition comprising a cannabinoid and at least one additional active agent selected from the group consisting of a vasoconstrictor and an astringent.

2. The composition according to claim 1, further comprising an anesthetic.

3. The composition according to claim 1 wherein the astringent is selected from the group consisting of: witch hazel, calamine and tannic acid.

4. The composition according to claim 3 wherein the astringent is witch hazel, and is present in an amount of between 40% and 60% of the liquid composition.

5. (canceled)

6. (canceled)

7. The composition according to claim 1 wherein the vasoconstrictor is selected from the group consisting of phenylephrine, ephedrine sulfate, epinephrine, or salts thereof.

8. The composition according to claim 7 wherein the vacoconstrictor is phenylephrine and is present in an amount between 0.1% and 1% of the liquid composition.

9. (canceled)

10. The composition according to claim 8 wherein phenylephrine is present in an amount of 0.25% of the liquid composition.

11. The composition according to claim 2 wherein the anesthetic is selected from the group consisting of: pramoxine, camphor, juniper tar, menthol, diperodon, phenacaine, benzocaine, benzyl alcohol, dibucaine, dyclonine, lidocaine, and tetracaine, and salts thereof.

12. The compositions according to claim 11 wherein the anesthetic is pramoxine, and is present in an amount between 0.1% and 2% of the liquid composition.

13. (canceled)

14. (canceled)

15. The composition according to claim 1 wherein the cannabinoid is cannabidiol (CBD).

16. The composition according to claim 15 wherein the CBD is present in an amount between 0.01% and 10% of the liquid composition.

17. The composition according to claim 16 wherein the CBD is present in an amount between 0.1% and 1% of the liquid composition.

18. The composition according to claim 17 wherein the CBD is present in an amount between 0.25% and 0.5% of the liquid composition.

19. (canceled)

20. The composition according to claim 1, further comprising an analgesic or antipruritic agent.

21. The composition according to claim 20 wherein the analgesic or antipruritic agent is selected from the group consisting of: camphor, juniper tar and menthol.

22. The composition according to claim 1, further comprising one or more of the following ingredients: water, a solvent, an emulsifier, an emollient, a moisturizer, a pH adjustment agent, a polymer, a humectant, an occlusive agent, a preservative, a thickener, an anti-irritation agent, a conditioning agent, a buffer, a vitamin, an extract, a natural oil, a wax, a penetration enhancer, a peptide, a sugar derivative, a fatty acid, a fatty alcohol, a silicone, a polyethyl-glycol, a fragrance, a pigment, an ester, a triglyceride, a butter, hyaluronic acid, and an absorbing powder.

23. (canceled)

24. (canceled)

25. The composition according to claim 1, wherein the liquid is an oil in water emulsion.

26. (canceled)

27. The composition according to claim 1, wherein the ratio between fabric and liquid composition is between 1:2 and 1:4.

28. The composition according to claim 27 wherein the ratio between fabric and liquid composition is 1:3.

29. A method for treatment of hemorrhoids comprising applying to an affect area of a subject in need thereof, a wet wipe according to claim 1.

30. (canceled)
